(19) United States
(12) Patent Application Publication
(10) Pub. No.: US 2004/0081679 A1
(43) Pub. Date: Apr. 29, 2004

(54) Wipe

(75) Inventors: Pascal Simon, Vitry Sur Seine (FR); Anne-Clotilde Roche, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/662,456

(22) Filed: Sep. 16, 2003

Related U.S. Application Data
(60) Provisional application No. 60/416,239, filed on Oct. 7, 2002.

(30) Foreign Application Priority Data
Sep. 19, 2002 (FR) 0211607

(51) Int. Cl. A61K 9/70

(52) U.S. Cl. 424/443

(57) ABSTRACT

The invention relates to an article containing a water-insoluble substrate and a substantially anhydrous liquid composition containing oil(s), emulsifying surfactant(s) and an aqueous gelling agent(s). After moistening, the article generates an unctuous cream having a viscosity such that it cannot be directly impregnated into the substrate by conventional means. This article constitutes in particular a wipe which may be used in particular for caring for, cleansing and/or removing makeup from the skin of the face and/or of the body, and for removing makeup from the eyes.

### REFERENCE TO PRIOR APPLICATIONS

This application claims priority to U.S. provisional application 60/416,239 filed Oct. 7, 2002, and to French patent application 0211607 filed Sep. 19, 2002, both incorporated herein by reference.

### FIELD OF THE INVENTION

The invention relates in general to an article comprising a water-insoluble substrate and a substantially anhydrous liquid composition comprising an oil, an emulsifying surfactant and an aqueous gelling agent. In a preferred embodiment the article, after moistening, generates an unctuous cream having a viscosity such that it cannot be directly impregnated into the substrate by conventional means. The invention article constitutes in particular a wipe, which may be used in particular for caring for, cleansing and/or removing makeup from, for example, the skin of the face and/or of the body, and for removing makeup from the eyes.

### BACKGROUND OF THE INVENTION

Cosmetic wipes generally consist of a substrate made of a material of natural or synthetic origin, which is preferably a nonwoven, but which may also be a mousse or a fabric, the substrate being impregnated with a composition suitable for the desired purpose, for example cleansing or removing makeup from the skin, or alternatively caring for the skin. These wipes are commonly used and are appreciated for their practical feature because they are disposable and are impregnated with the required and sufficient quantity of cleansing or treatment product. The use of these wipes avoids handling and transporting bottles containing lotions or milk.

The impregnated wipes may be wet or dry. Dry wipes should be moistened before use and may for example be impregnated with a foaming composition which generates foam when the wipe is moistened, as described for example in the document U.S. Pat. No. 4,303,543. The moist wipes may be impregnated with an aqueous composition such as a makeup removing lotion or a makeup removing milk for example, and they are directly applied to the face or the body. They may also be impregnated with an anhydrous composition containing for example a mixture of oils and surfactants, and the wipe is then either directly used on the face or the body, or moistened beforehand with a small amount of water in order to emulsify the oil/surfactant mixture before applying to the skin, as described for example in the document U.S. Pat. No. 6,136,775.

The impregnation of the substrates with the impregnating composition may be carried out according to different techniques, such as spraying or dipping. However, these techniques can only be used if the impregnating compositions are sufficiently fluid and have a viscosity close to that of water. Indeed, it is not possible to correctly moisten the substrate when the compositions are too viscous; the substrate is then incorrectly impregnated and, furthermore, it is then difficult to cut it, to fold it and to package it in bags. In addition, the article obtained is unpleasant to use because the impregnating product remains at the surface of the substrate or does not impregnate it homogeneously, such that certain zones of the article contain too much product, and others are free of it or contain too little of it.

Thus, the compositions suitable for impregnating the substrates of articles and in particular of wipes are always fluid; they are generally aqueous or aqueous-alcoholic lotions, or fluid oil-in-water (O/W) emulsions which do not contain or contain very little gelling agent and a very small dispersed oily fraction, because the increase in the dispersed oily fraction in an O/W emulsion increases the viscosity of the composition and thus makes its use for the manufacture of a wipe difficult. Thus, the documents WO-A-99/13861 and WO-A-01/35924 describe substantially dry articles, of which impregnating compositions contain very not much oil. Furthermore, this oil is added directly on the cloth which is impregnated with a composition of foaming surfactant, that may provide a default of homogeneity in the article.

The wipes comprising an oil-based composition may be used for skin care (impregnation of oils for the body) or for makeup removal from the skin (impregnation of makeup removing oils), but during application to the skin, they leave a fatty film which the wipe, which is itself impregnated with oil, cannot remove. This is all the more unpleasant to the eye when the wipe is used for removing makeup from the eyes. Moreover, the wipes containing oils combined with surfactants are used after moistening with a small amount of water, the addition of water making it possible to obtain an emulsion by dispersing the mixture of oil and of surfactant in the water, but the emulsion thus obtained remains very liquid, without consistency and tends to flow out of the substrate.

Thus, the compositions for impregnating the wipes used up until now lack consistency and do not make it possible to obtain a creamy texture, which criteria are important for obtaining good comfort during use both in caring for and in removing makeup from the skin. However, the comfort provided by a cosmetic product, during its use and immediately after its use, is as important as its efficacy. The fact that a cream and not a fluid is obtained provides comfort for the skin which is highly appreciable.

Thus, the need remains to have an article (wipe or compress or foam, etc.) which can give, during application to the skin, a thick and unctuous composition, this article being both easy to use and comfortable upon application.

### DETAILED DESCRIPTION OF THE PREFERRED EMBODIMENTS

The inventors have found, surprisingly, a substantially anhydrous composition containing one or more oils, one or more solubilized emulsifying surfactants, and one or more hydrophilic gelling agents that satisfies the above-described objects and desires of the art. Preferably, the invention composition is sufficiently fluid to be able to be impregnated by conventional means into an article such as a wipe or compress, or any other preferably absorbent substrate which is sufficiently resistant so as not to disintegrate during its use on the skin, and is capable of generating a thick creamy texture during moistening with a small amount of water. Thus, from this substantially anhydrous composition of the invention, a means has been provided for obtaining a wipe generating a composition having a thick texture similar to that of a cream, which composition it would have been impossible to impregnate directly into a wipe by conventional means.

The composition according to the invention presents the advantage of being substantially anhydrous, and containing one or more hydrophilic gelling agents that are preferably in a dispersed state, and of being impregnated on a substrate if desired thus providing a substantially dry article, while generally, dry articles are obtained by impregnating with an aqueous composition and drying, as described for example in the document WO-A-99/13861.

One subject of the present invention is therefore an article containing (A) a water-insoluble substrate comprising one or more layers, and (B) a substantially anhydrous composition which can be in contact therewith, said substantially anhydrous composition comprising a.) at least 10% by weight of one or more oils, relative to the total weight of the composition, b.) at least one emulsifying surfactant, and c.) at least one hydrophilic gelling agent. The substantially anhydrous composition can be added to or impregnated into the substrate, for example, to provide contact.

The obtained article is substantially dry, meaning that it contains generally less than 10% by weight of water, preferably less than 5% by weight of water relative to the total weight of the composition.

The expression “substantially anhydrous” is understood to mean a composition containing less than 10% by weight of water, and preferably less than 5% by weight of water, relative to the total weight of the composition. The term “anhydrous,” by itself, means a lack of water. The quantity of water in the composition can preferably range from 0 to 10% and more preferably from 0 to 5% of the total weight of the composition.

The substantially anhydrous composition of the invention is preferably liquid. Preferably, the composition generally has a viscosity of less than 150 mPa.s and more preferably of less than 100 mPa.s. This viscosity preferably ranges from 1 mPa.s to 100 mPa.s, measured at room temperature (25° C.) with a RHEOMAT RM 180 apparatus, rotor 1 or 2 depending on the viscosity of the liquid.

The article according to the invention has the advantage of being very easy to handle, because to provide beneficial effects it can, for example, simply be moistened with a small amount of water, slightly pressed between the fingers in order to cause water to penetrate and thus emulsify the composition impregnated with water. A composition having a creamy texture is then formed which is very pleasant as regards its appearance, its feel, and also during its application to the skin. In addition, the user can adjust at will the viscosity of the cream by adding more or less water to the impregnated substrate.

The article according to the invention is preferably a cosmetic article which is appropriate for caring for and/or treating the skin and for cleansing or removing makeup from the skin of the face and/or of the body and/or of the eyes. It can in particular constitute a wipe, but it can also be in the form of a glove, a mitten or in any other form appropriate for use for example on the face or the body.

Another subject of the invention is the use of the article as defined above, for example for caring for, cleansing and/or removing makeup from the skin and/or the eyes.

The composition according to the invention preferably contains a physiologically acceptable medium, that is to say a medium which is compatible with the skin, the mucous membranes, the hair and the scalp.

Another advantage of the article according to the invention results from the fact that it does not contain water or that it contains very little of it. As a result, it is not absolutely necessary to introduce preservatives in order to protect the formula. These wipes may therefore be advantageously free of preservatives, and may be used more particularly for sensitive skins.

Accordingly, a subject of the invention is also the cosmetic use of the article as defined above, for caring for, cleansing and/or removing makeup from sensitive skins and/or sensitive eyes.

I. Hydrophilic Gelling Agents

The expression “hydrophilic agent” is understood to mean an agent which is soluble or dispersible in water.

The expression “gelling agent” is understood to mean an agent which increases the viscosity of an aqueous composition containing it.

The total quantity of hydrophilic gelling agent in the composition of the invention is not particularly limited and can depend on the gelling agent used. The amount should be such that the composition is liquid and therefore has a viscosity of less than 150 mPa.s. This quantity may range, for example, from 0.1 to 20% by weight (of active substance), preferably from 0.5 to 15% by weight, even better from 1 to 10%, better still from 1 to 6% by weight, and more preferably from 2 to 6% by weight relative to the total weight of the composition.

It is possible to use any type of hydrophilic gelling agent. As noted throughout, more than one such agent may be used in the composition of the invention. Although hydrophilic gelling agents make it possible to obtain a creamy texture after moistening, and thus are useful herein, a more highly preferred gelling agent is one capable of practically instantly and homogeneously thickening the composition after moistening the article (e.g., wipe), this being in order to avoid the user having to handle the wipe for too long, and having to wait for too long for the wipe, after moistening, to give a thickened creamy composition.

According to a preferred embodiment of the invention, the hydrophilic gelling agent comprises a hydrophilic polymer.

It is possible to use in particular polymers provided in the form of a powder or in the form of a water-in-oil (W/O) emulsion (inverse emulsion) containing little water and such that the final composition contains less than 10% of water and preferably less than 5% of water. Preferably, polymers provided in the form of W/O emulsions are used.

Moreover, to allow homogeneous impregnation of the composition into the substrate, it is preferable to obtain a dispersion of the polymer in oil which is relatively stable to sedimentation. However, if the dispersion of polymer in oil is not perfectly stable, it is possible to carry out the preparation of the composition and its impregnation into/contact with the substrate continuously, or also to stir the vessel containing the composition in order to homogenize it just before the impregnation of the substrate.

Hydrophilic polymers useful as hydrophilic gelling agents include crosslinked polymers of acrylic acid or methacrylic acid, polymers of 2acrylamido-2-methylpropanesulphonic acid, crosslinked copolymers of acrylamide and 2-acrylamido-2-methylpropanesulphonic acid, and mixtures thereof. These polymers are capable of practically instantly and homogeneously thickening the composition after moistening a, e,g, wipe/composition combination.

These crosslinked copolymers and homopolymers are preferably crosslinked with a crosslinking agent which may be in particular a compound having an olefinic polyunsaturation such as those selected from the group comprising divinylbenzene, tetraallyloxyethane, methylenebisacrylamide, diallyl ether, polyallylpolyglyceryl ethers or allyl ethers of an alcohol of the sugar series, such as erythritol, pentaerythritol, allylpentaerythritol, arabitol, mannitol, sorbitol, allylsucrose or glucose. Preferentially, methylenebisacrylamide is used as crosslinking agent. Preferably, the crosslinking agent is present in the polymer or the copolymer in a quantity ranging from 0.06 to 1 millimol per mol of monomer or of the mixture of monomers.

Useful crosslinked homopolymers of acrylic acid or methacrylic include those marketed under the names Carbopol 940, Carbopol 941, Carbopol 980, Carbopol 981, Carbopol ETD 2001, Carbopol ETD 2020, Carbopol ETD 2050, Carbopol 2984, Carbopol 5984, Carbopol Ultrez 10 by the company Goodrich, those marketed under the names Synthalen K, Synthalen L and Synthalen M by the company 3V; those marketed under the names Modarez V1250 PX, Modarez V2000 PX, Viscaron A 1600 PE, Viscaron A700 PE by the company Protex.

As these polymers are anionic, a neutralizing agent is preferably added to the composition. The required quantity of neutralizing agent (base) is introduced into the oily composition, preferably in the form of an inorganic or organic base such as sodium hydroxide, potassium hydroxide, aqueous ammonia or an amine such as triethanolamine or monoethanolamine, or mixtures thereof. Preferably, the neutralizing agent is a liquid amine such as triethanolamine for example which is easily solubilized in the oily mixture.

The 2-acrylamido-2-methylpropanesulphonic acid polymers may be optionally crosslinked with the crosslinking agents indicated above and/or neutralized with the bases described above. They are preferably crosslinked and at least partially neutralized. As polymers of 2-acrylamido-2-methylpropanesulphonic acid, there may be mentioned for example the poly(2-acrylamido-2-methylpropanesulphonic acid) marketed by the company Hoechst under the tradename “Hostacerin AMPS” (CTFA name: ammonium polyacryldimethyltauramide).

The crosslinked copolymers of acrylamide and 2-acrylamido-2-methylpropanesulphonic acid (AMPS) preferred are those obtained by copolymerization, by the free radical route, of 15-85 mol % of acrylamide and 15-85-mol % of 2-acrylamido-2-methylpropanesulphonic acid, in particular 30-70 mol % of acrylamide and 30-70 mol % of 2-acrylamido-2-methylpropanesulphonic acid, and better still 55-70 mol % of acrylamide and 30-45 mol % of 2-acrylamido-2-methylpropanesulphonic acid.

Moreover, the 2-acrylamido-2-methylpropanesulphonic acid may be at least partially neutralized in the form of a salt, for example with sodium hydroxide, with potassium hydroxide, or with a low-molecular weight amine such as triethanolamine or monoethanolamine, or mixtures thereof. Preferably, the neutralizing agent is a liquid amine such as triethanolamine for example which is easily solubilized in the oily mixture.

According to a particular embodiment of the invention, the crosslinked anionic copolymer of acrylamide and AMPS used in the composition of the invention is provided in the form of a W/O emulsion. There may be mentioned for example the W/O emulsion containing about 32% of water, from 35 to 40% by weight of the copolymer, from 15 to 25% by weight of a mixture of C₁₂-C₁₃ isoparaffin hydrocarbons, from 3 to 8% by weight of a polyoxyethylenated surfactant such as polyethylene glycol lauryl ether containing 7 moles of ethylene oxide, and emulsion marketed under the name SEPIGEL 305 (C.T.F.A. name: polyacrylamide/C13-14 isoparaffin/laureth 7) by the company SEPPIC, and the emulsion containing 40% of copolymer and 30% of water, marketed under the name SIMULGEL 600 (C.T.F.A. name: acrylamide/sodium acryloyldimethyltaurate copolymer/isohexadecane/polysorbate 80) by the company SEPPIC.

These copolymers which are provided in the form of a W/O emulsion have the advantage of dispersing very well in the mixture of oils and surfactants and of practically instantly giving a creamy and thick texture after moistening the wipe. Moreover, these copolymers have the advantage of making it possible, after moistening the article according to the invention, to obtain a composition which, depending on the greater or lesser quantity of water introduced into the article, may be provided in the form of a water-in-oil (W/O) inverse emulsion, or of an oil-in-water (O/W) direct emulsion when the quantity of water introduced is greater. The obtaining of a W/O emulsion allows an easier makeup removal because the oily phase is external, the presence of oils increasing the efficiency of the removal of the oily products present on the skin, in particular of makeup products. When the quantity of water is greater and an O/W emulsion is obtained, the removal of the makeup or the rinsing of the skin are facilitated by the presence of water in the external phase of the emulsion. However, the composition according to the invention, as noted above, preferably contains at the most 10% by weight of water.

II. Oils

The composition contains at least 10% by weight of one or more oils, relative to the total weight of the composition. It may contain one or more oils, in particular cosmetic oils. The quantity of oil(s) is not particularly limited and may range for example from 10 to 99% by weight, preferably from 30 to 90% by weight, and even better from 40 to 85% by weight relative to the total weight of the composition.

Useful oils which can be used in the composition of the invention include for example:

hydrocarbon oils of animal origin, such as perhydrosqualene;

hydrocarbon oils of plant origin, such as liquid triglycerides of fatty acids containing from 4 to 10 carbon atoms such as triglycerides of heptanoic or octanoic acids or alternatively, for example, sweet almond, sunflower, maize, soyabean, coriander, gourd, grapeseed, sesame, hazelnut, apricot, macadamia, arara, castor or avocado oils, triglycerides of caprylic/capric acids such as those sold by the company Stearineries Dubois or those sold under the names Miglyol 810, 812 and 818 by the company Dynamit Nobel, jojoba oil, shea butter oil;

synthetic esters and ethers, in particular of fatty acids, such as the oils of formulae R¹COOR² and R¹OR² in which R¹ represents the residue of a fatty acid containing from 8 to 29 carbon atoms, and R² represents a branched or unbranched hydrocarbon chain containing from 3 to 30 carbon atoms, such as for example Purcellin oil, isononyl isononanoate, isopropyl myristate, isopropyl palmitate, 2-ethylhexyl palmitate (or octyl palmitate), 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates, decanoates of fatty alcohols; polyol esters, such as propylene glycol dioctanoate, neopentyl glycol diheptanoate and diethylene glycol diisononanoate; and the pentaerythritol esters such as pentaerythrityl tetraisostearate;

linear or branched hydrocarbons of mineral or synthetic origin, such as volatile or nonvolatile paraffin oils and derivatives thereof, petroleum jelly, polydecenes, hydrogenated polyisobutene such as Parleam® oil;

fatty alcohols having from 8 to 26 carbon atoms, such as cetyl alcohol, stearyl alcohol and their mixtures (cetearyl alcohol), octyldodecanol, 2-butyloctanol, 2-hexyldecanol, 2-undecylpentadecanol, oleyl alcohol or linoleyl alcohol;

alkoxylated and in particular ethoxylated fatty alcohols such as oleth-12 or ceteareth-20;

partially hydrocarbonaceous and/or siliconized fluorinated oils such as those described in the document JP-A-2-295912. As fluorinated oils, there may also be mentioned perfluoromethylcyclopentane and perfluoro-1,3-dimethylcyclohexane which are sold under the names “FLUTEC PC1®” and “FLUTEC PC3®” by the company BNFL Fluorochemicals; perfluoro-1,2-dimethylcyclobutane; perfluoroalkanes such as dodecafluoropentane and tetradecafluorohexane, which are sold under the names “PF 5050®” and “PF 5060®” by the company 3M or alternatively bromoperfluorooctyl sold under the name “FORALKYL®” by the company Atochem; nonafluoromethoxybutane sold under the name “MSX 4518®” by the company 3M and nonafluoroethoxyisobutane; perfluoromorpholine derivatives such as 4-trifluoromethylperfluoromorpholine sold under the name “PF 5052®” by the company 3M;

silicone oils such as volatile or nonvolatile polymethylsiloxanes (PDMS) having a linear or cyclic silicone chain, which are liquid or pasty at room temperature, in particular cyclopolydimethylsiloxanes (cyclomethicones) such as cyclohexasiloxane; polydimethylsiloxanes containing alkyl, alkoxy or phenyl groups, which are pendant or at the end of a silicone chain, groups having from 2 to 24 carbon atoms; phenylated silicones such as phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxysilicates and polymethylphenylsiloxanes;

mixtures thereof.

The expression “hydrocarbon oil” is understood to mean in the list of oils mentioned above, any oil predominantly containing carbon and hydrogen atoms, and optionally ester, ether, fluorinated, carboxylic acid and/or alcohol groups.

III. Surfactants

The composition comprises one or more emulsifying surfactants. These surfactants preferably allow that, after moistening the article with water, oil is emulsified with water by giving a cream. The quantity of surfactant(s) is not particularly limited and may range for example from 0.1 to 90% by weight, preferably from 1 to 60% by weight, even better from 5 to 40% by weight and more still better from 5 to 30% by weight relative to the total weight of the composition.

The surfactant or the mixture of surfactants preferably has an HLB (Hydrophilic Lipophilic Balance) ranging from 5 to 15, more preferably from 8 to 14, and should be soluble in the oily phase.

These surfactants may be nonionic, anionic, amphoteric or zwitterionic.

Preferably, the surfactant(s) are selected from the group consisting of nonionic surfactants. Useful nonionic surfactants include for example fatty acid esters of polyols and their oxyalkylenated and in particular oxyethylenated derivatives; fatty alcohol ethers of polyols and their oxyalkylenated and in particular oxyethylenated derivatives, and mixtures thereof. In the case of oxyalkylenated fatty acid esters of polyols or of oxyalkylenated fatty alcohol ethers of polyols, there may be for example from 1 to 150 oxyalkylenated and in particular oxyethylenated groups, and preferably from 2 to 100 oxyalkylenated and in particular oxyethylenated groups.

As surfactants of this type, there may be mentioned more particularly:

oxyethylenated or nonoxyethylenated, preferably oxyethylenated, sorbitan fatty acid esters such as (CTFA name) Polysorbate 65, Polysorbate 85, PEG-5 Sorbitan Isostearate, PEG-20 Sorbitan Triisostearate, PEG-20 Sorbitan Isostearate, PEG-40 Sorbitan Septaoleate, PEG-20 Sorbitan Tetraoleate, PEG-20 Sorbitan Trioleate;

oxyethylenated or nonoxyethylenated, preferably oxyethylenated, glyceryl fatty acid esters such as (CTFA name) PEG-20 glyceryl triisostearate, PEG-7 glyceryl cocoate;

polyglyceryl fatty acid esters such as (CTFA name) polyglyceryl-3 triisostearate, polyglyceryl-10 diisostearate, polyglyceryl-6 isostearate, polyglyceryl-3 diisostearate, polyglyceryl-10 trioleate, polyglyceryl-10 trilaurate;

polyethylene glycol fatty acid esters such as (CTFA name) PEG-8 stearate, PEG-6 oleate, PEG-6 isostearate, PEG-12 isostearate, PEG-12 diisostearate, PEG-8 isostearate, PEG-8 diisostearate, PEG-10 isostearate;

polyoxyethylenated and/or polyoxypropylenated fatty alcohol ethers such as for example ceteareth-12 and ceteareth-20 (CTFA name), and mixtures containing them, such as the mixture marketed under the name Emulgade CM by the company Henkel (mixture of cetearyl isononanoate, ceteareth-20, cetearyl alcohol, glyceryl stearate, glycerin, ceteareth-12 and cetyl palmitate)

and mixtures thereof.

It is also possible to use surfactants having an HLB greater than 15, as long as one or more other surfactants are added thereto so that the HLB of the mixture ranges from 5 to 15. Thus, it is possible to use for example PEG-40 Stearate (HLB 16.9) as a mixture with another surfactant having an HLB such that the mixture has an HLB of 5 to 15.

It is also possible to add foaming surfactants, especially for articles, in particular wipes, for cleansing or removing makeup from the skin. As surfactants of this type, useful examples include:

(1) among the nonionic surfactants, oxyethylenated oxypropylenated block polymers such as Poloxamer 184 (CTFA name); alkyl polyglycosides and in particular alkyl polyglucosides (APG) having an alkyl group containing from 6 to 30 carbon atoms (alkyl-C₆-C₃₀ polyglucosides) and preferably 8 to 16 carbon atoms, such as for example decyl glucoside (C9/C11 alkyl polyglucoside (1.4) such as the product marketed under the name MYDOL 10 by the company Kao Chemicals, the product marketed under the name PLANTAREN 2000 UP or PLANTACARE 2000 UP by the company Henkel, and the product marketed under the name ORAMIX NS 10 by the company Seppic; caprylyl/capryl glucoside such as the product marketed under the name ORAMIX CG 110 by the company Seppic; lauryl glucoside such as the products marketed under the names PLANTAREN 1200 N and PLANTACARE 1200 by the company Henkel; and cocoglucoside such as the product marketed under the name PLANTACARE 818/UP by the company Henkel;

(2) among the anionic surfactants, alkyl sulphates, alkyl ether sulphates and their salts, in particular their sodium salts, such as the mixture of Sodium Laureth Sulphate/Magnesium Laureth Sulphate/Sodium Laureth-8 Sulphate/Magnesium Laureth-8 Sulphate, sold under the name Texapon ASV by the company Henkel; sodium lauryl ether sulphate (C12-14 70/30) (2.2 EO) marketed under the names SIPON AOS 225 or TEXAPON N702 PATE by the company Henkel, ammonium lauryl ether sulphate (C12-14 70/30) (3 EO) marketed under the name SIPON LEA 370 by the company Henkel; ammonium (C12-C14) alkyl ether (9 EO) sulphate marketed under the name RHODAPEX AB/20 by the company Rhodia Chimie;

(3) among amphoteric or zwitterionic surfactants, alkylamido alkylamine derivatives such as N-disodium N-cocoyl-N-carboxymethoxyethyl-N-carboxymethylethylenediamine (CTFA name: disodium cocoampho-diacetate) marketed as an aqueous saline solution under the name MIRANOL C2M CONC NP by the company Rhodia Chimie; N-sodium N-cocoyl-N-hydroxyethyl-N-carboxymethylethylenediamine (CTFA name: sodium cocamphoacetate) and the mixture of coconut acid ethanolamides (CTFA name: Cocamide DEA).

According to a preferred embodiment of the invention, the composition comprises (i) from 1 to 6% by weight of one or more hydrophilic gelling agents selected from the group consisting of crosslinked polymers of acrylic acid or methacrylic acid, polymers of 2-acrylamido-2-methylpropanesulphonic acid, crosslinked copolymers of acrylamide and 2-acrylamido-2-methylpropanesulphonic acid; (ii) from 30 to 90% by weight of one or more oils; and (iii) from 5 to 40% by weight of one or more emulsifying surfactants selected from the group consisting of fatty acid esters of polyols and their oxyalkylenated derivatives; fatty alcohol ethers of polyols and their oxyalkylenated derivatives. This composition is preferably impregnated on/into the article of the invention.

IV. Additives

The invention composition may additionally comprise additives, materials, etc. not specifically listed above. One such class of additives is adjuvants conventionally used in the cosmetic or dermatological field. Useful adjuvants include those selected from the group consisting of organic solvents, emollients, antioxidants, chelators, perfumes, UV-screening agents, colouring matter, hydrophilic or lipophilic active agents, lipophilic gelling agents, preservatives, lipid vesicles which may optionally encapsulate one or more active agents, or any other ingredient customarily used in cosmetics or dermatology, and mixtures thereof. The quantities of the various additives may be those conventionally used. Of course, the nature and quantity of these additives is preferably such that they do not impair the composition according to the invention. The quantity of these additives may range for example from 0.01 to 30% by weight relative to the total weight of the composition.

The adjuvants may be chosen in particular from lipophilic or hydrophilic active agents.

Useful active agents include for example antiseborrhoeic active agents which allow cleansing of the excess sebum on the skin, and antimicrobial agents which remove from the skin the microorganisms which may be present on it, and mixtures of these active agents.

As antiseborrhoeic active agents, there may be mentioned for example sulphur and sulphur-containing derivatives, benzoyl peroxide, zinc derivatives such as zinc sulphate and zinc oxide, aluminium chloride, selenium disulphide, B vitamins and in particular panthenol (vitamin B5) and niacinamide (vitamin B6 or PP), and mixtures thereof.

As antimicrobials, the following active agents may be mentioned for example: β-lactam derivatives, quinolone derivatives, ciprofloxacin, norfloxacin, tetracycline and its salts (hydrochloride), erythromycin and its salts (zinc, estolate or stearate salt), amikacin and its salts (sulphate), 2,4,4′-trichloro-2′-hydroxydiphenyl ether (triclosan), 3,4,4′-trichlorobanilide (tricarban), phenoxyethanol, phenoxypropanol, phenoxyisopropanol, doxycycline and its salts (hydrochloride), capreomycin and its salts (sulphate), chlorhexidine and its salts (gluconate, hydrochloride), chlorotetracycline and its salts (hydrochloride), oxytetracycline and its salts (hydrochloride), clindamycin and its salts (hydrochloride), ethambutol and its salts (hydrochloride), hexamidine and its salts (isethionate), metronidazole and its salts (hydrochloride), pentamidine and its salts (hydrochloride), gentamycin and its salts (sulphate), kanamycin and its salts (sulphate), lineomycin and its salts (hydrochloride), methacycline and its salts (hydrochloride), methenamine and its salts (hippurate, mandelate), minocycline and its salts (hydrochloride), neomycin and its salts (sulphate), netilmicin and its salts (sulphate), paromomycin and its salts (sulphate), streptomycin and its salts (sulphate), tobramycin and its salts (sulphate), miconazole and its salts (hydrochloride), amanfadine and its salts (sulphate, hydrochloride), octopirox, para-chloro-meta-xylenol, nystatin, tolnaftate, zinc pyrithione, clotrimazole, salicylic acid, 5-n-octanoylsalicylic acid (or capryloylsalicylic acid), benzoyl peroxide, 3-hydroxybenzoic acid, glycolic acid, lactic acid, 4-hydroxybenzoic acid, acetylsalicylic acid, 2-hydroxybutanoic acid, 2-hydroxypentanoic acid, 2-hydroxyhexanoic acid, phytic acid, N-acetyl-L-cysteine acid, lipoic acid, azelaic acid, arachidonic acid, ibuprofen, naproxen, hydrocortisone, acetominophen, resorcinol, lidocaine hydrochloride, neomycin sulphate, octoxyglycerol, octanoylglycine (or capryloylglycine), caprylylglycol (1,2-octanediol) and 10-hydroxy-2-decanoic acid, and mixtures thereof.

There may also be mentioned as active agents, without this list being limiting, α-hydroxy acids such as lactic acid, glycolic acid, citric acid, and their derivatives; essential oils; vitamins and in particular retinol (vitamin A), ascorbic acid (vitamin C), tocopherol (vitamin E), panthenol (vitamin B5) and their derivatives (esters for example); coenzymes and in particular coenzyme Q10 or ubiquinone; enzymes such as for example lipases, proteases, phospholipases, cellulases, peroxidases, in particular lactoperoxidases, catalases, superoxide dismutases and plant extracts containing the abovementioned enzymes; yeasts such as Saccharomyces Cerevisiae; steroids, antioxidants and anti-free radicals; moisturizers such as polyols (glycerin, sorbitol, sugars), protein hydrolysates, urea and mixtures containing it; antielastase and anticollagenase agents; plant extracts and in particular plankton extracts; and mixtures thereof.

As examples of steroids, there may be mentioned dehydroepiandrosterone (or DHEA), and (1) its biological precursors and derivatives, in particular DHEA salts and esters such as DHEA sulphate and salicylate, 7-hydroxyDHEA, 7-ketoDHEA, 7-hydroxy- and 7-ketoDHEA esters, in particular 3-beta-acetoxy-7-oxo DHEA, and (2) its chemical precursors and derivatives, in particular sapogenins such as diosgenin or hecogenin, and/or their derivatives such as hecogenin acetate, and/or natural extracts containing them and in particular extracts of Dioscorea, such as wild yam.

The lipophilic adjuvants may be dissolved directly in the oils, while the hydrophilic adjuvants may be dispersed in the composition with the aid of the surfactants present.

Preferably, the composition according to the invention contains at least one polyol such as glycerin, where the amount of polyol may range for example from 0.5 to 10% by weight and better from 2 to 10% by weight relative to the total weight of the composition.

It is possible to add also to the composition according to the invention a lipophilic gelling agent provided that it does not thicken the impregnating composition before impregnating into the substrate. The introduction of such a gelling agent makes it possible to obtain a film-forming feel when the moist article is applied to the skin. As lipophilic gelling agents, there may be mentioned for example the dextrin palmitate marketed under the name RHEOPEARL TL by the company Chiba Flour Milling.

Compositions according to the invention may be prepared according to the following method: the procedure starts with preparing the mixture of oils, and then the surfactants are incorporated therein at room temperature or in the hot state depending on whether they are in liquid or in solid form, and the hydrophilic gelling agent and the adjuvants are then incorporated into the mixture obtained.

Substrate

The water-insoluble substrate may comprise one or more layers and it may be selected from the non-limiting group consisting of woven materials, nonwoven materials, foams, sponges, cotton wool, in the form of sheets, balls or films. It may be in particular a nonwoven substrate based on fibres of natural origin (e.g., flax, wool, cotton, silk) or of synthetic origin (e.g., cellulose derivatives, viscose, polyvinyl derivatives, polyesters such as polyethylene terephthalate, polyolefins such as polyethylene or polypropylene, polyamides such as Nylon, acrylic derivatives). The nonwoven materials are described in general in RIEDEL “Nonwoven Bonding Methods & Materials”, Nonwoven World (1987). These substrates are obtained according to the customary methods of the technique for preparing nonwoven materials. In preferred embodiments the substrate takes the form of a wipe or compress, or foam (e.g., polyurethane foam) sheet, etc.

When the substrate is a nonwoven material, a thick nonwoven material is preferably used which does not form a ball and which is fairly solid so as not to disintegrate and not to become fluffy during application to the skin. It should be absorbent, soft at least on one side for removing makeup from the eyes in particular. As appropriate nonwoven materials, there may be mentioned for example those marketed under the names Ultraloft 15285-01, Ultraloft 182-008, Ultraloft 182-010, Ultraloft 182-016 by the company BBA, Vilmed M1519 Blau, Vilmed M 1550 N and 112-132-3 by the company Freudenberg, that marketed under the name Norafin 11601-010B by the company Jacob Holm Industries, the flocked nonwoven materials marketed under the names Univel 109 and Univel 119 by the company Uni Flockage.

This substrate may contain one or more layers having identical or different properties and may have properties of elasticity, softness and the like which are appropriate for the desired use. The substrates may contain for example two parts having different elasticity properties as described in the document WO-A-99/13861 or may contain a single layer with different densities as described in the document WO-A-99/25318 or may contain two layers of different textures as described in the document WO-A-98/18441.

In addition, when the article is used for the body, the substrate may comprise at least one rough side in order to allow massage of the skin at the same time.

Moreover, the substrate may also advantageously have a sufficient water absorbing capacity in order to dry the body, this absorption capacity preferably ranging from 800% to 3000%. In this precise case, the nonwoven materials used preferably have a high weight (weight>90 g/m² ) and a high thickness, or alternatively they contain at least one superabsorbant polymer. As nonwoven materials having a high weight, there may be mentioned for example those marketed under the name Aquadim V100 by the company Tharreau, that marketed under the name Norafin 1.111.00.01 by the company Jacob Holm, that marketed under the name 112/132/4 by the company Freudenberg. As nonwoven materials containing a superabsorbant polymer, there may be mentioned those marketed under the names Dritex 120NN42 and 13OWNNF60 by the company Georgia Pacific, and those marketed under the names HY0101046 or HY0301038 by the company BBA.

The substrate may be of any size and any shape and is preferably tailored in a manner appropriate for the desired aim. It may thus have for example the shape of a rectangular wipe, or the shape of a glove or a mitten, which are easy to wear over the hand, or the shape of a round compress. It may preferably have a surface area of between 0.005 m² and 0.1 m², more preferably between 0.01 m² and 0.05 m².

The level of impregnation of the composition into the substrate is not limited, and generally preferably ranges from 10 to 1500%, preferably from 50 to 500% and even better between 70 and 250%. The techniques for impregnating/contacting the substrates with compositions are well known in this field and are all applicable to the present invention. In general, the impregnating composition is added to the substrate by one or more techniques comprising immersion, coating, spraying and the like.

The subject of the invention is also a cosmetic method for caring for, cleansing and/or removing makeup from the skin and/or the eyes, consisting in passing over the skin and/or the eyes an article as defined above.

In a preferred embodiment the invention relates to an article containing a water-insoluble substrate and a substantially anhydrous liquid composition containing oil(s), emulsifying surfactant(s) and an aqueous gelling agent(s). After moistening, the article generates an unctuous cream having a viscosity such that it cannot be directly impregnated into the substrate by conventional means. This article constitutes in particular a wipe which may be used in particular for caring for, cleansing and/or removing makeup from the skin of the face and/or of the body, and for removing makeup from the eyes.

### EXAMPLES

The invention will now be illustrated by the following non-limiting examples. That is, the examples below are given by way of illustration and without limitation. The names are in the form of the chemical name or in the form of the CTFA name. The quantities therein are given in % by weight unless otherwise stated.

### Example 1

### Makeup Removing Compress

| Compounds | Quantities in % |
|---|---|
| Oil | |
| Ethylhexyl palmitate | 76.5 |
| PEG-20 glyceryl triisostearate | 8.5 |
| Surfactants | |
| PEG-20 glyceryl triisostearate | 8.5 |
| PEG-40 stearate | 2 |
| Moisturizing active agent | |
| Glycerin | 5 |
| Hydrophilic thickener | |
| Simulgel 600 (with 40% of polymer) (CTFA name: | 8 |
| acrylamide/sodium acryloyldimethyltaurate copolymer/ | (i.e. 3.2% |
| isohexadecane/polysorbate 80) | of polymer) |

The composition of example 1 is impregnated into a compress in the form of a round, oval, rectangular or square nonwoven material whose surface is suitable for removing makeup from the face, for example 0.0016 m² to 0.01 m². The impregnation rate is 100%.

Method of use: During use, the compress is taken in the hand, it is briefly passed under tapwater, and then it is passed over the face in order to remove makeup. On rubbing the compress on the face, the anhydrous composition instantly emulsifies with the added water to form a thick and unctuous makeup removing milk which is very comfortable and does not run. The face may then be rinsed with water, a tonic lotion, or allowed to dry.

### Example 2

### Care Wipe or Care Glove for the Body

| Compounds | Quantities in % |
|---|---|
| oils | |
| Ethylhexyl palmitate | 50 |
| Parleam oil | 25 |
| PEG-20 glyceryl triisostearate | 13 |
| Surfactants | |
| PEG-40 stearate | 2 |
| Active agents | |
| Glycerin | 5 |
| Hydrophilic thickener | |
| Simulgel 600 (with 40% of polymer) (CTFA name: | 5 |
| acrylamide/sodium acryloyldimethyltaurate copolymer/ | (i.e. 2% of |
| isohexadecane/polysorbate 80) | polymer) |

The composition is impregnated into a wipe whose surface is suitable for application to the entire surface of the body, for example 0.02 m² to 0.25 m². The impregnation rate is 200%.

Preferably, the substrate used for this example has the shape of a glove or a mitten, these shapes having the advantage of not slipping over the body as a wipe may do. The glove is preferably made of nonwoven material and consists for example of two sheets of nonwoven material cut to the desired shape and joined, stuck or sewn at the periphery. The method of using these gloves or mittens is simple: after a shower or a bath, the glove is passed over the wet skin, over the whole body. The passage of the glove over the wet skin emulsifies the oil contained in the glove and deposits in the form of an unctuous and soft moisturizing body milk.

Advantageously, the glove consists of a very absorbant nonwoven material, and it additionally has in this case the advantage of allowing the skin to be dried at the same time.

### Example 3

### Makeup Removing Compress

| Compounds | Quantities in % |
|---|---|
| Oils | |
| Parleam oil | 40 |
| Isododecane | 28 |
| Oil thickener | |
| Dextrin palmitate (Rheopearl TL) | 2 |
| Surfactant | |
| PEG-20 glyceryl tri-isostearate | 25 |
| Hydrophilic thickener | |
| Simulgel 600 (with 40% of polymer) (CTFA name: | 5 |
| acrylamide/sodium acryloyldimethyltaurate copolymer/ | (i.e. 2% of |
| isohexadecane/polysorbate 80) | polymer) |

The method of impregnation, the method of use and the substrate are identical to those of Example 1. The impregnation rate is 150%.

This compress generates a water-in-oil inverse emulsion after moistening.

The above written description of the invention provides a manner and process of making and using it such that any person skilled in this art is enabled to make and use the same, this enablement being provided in particular for the subject matter of the appended claims, which make up a part of the description herein, and including an article, more particularly a wipe, containing at least one water-insoluble substrate and an anhydrous liquid composition containing at least one oil, at least one emulsifying surfactant and at least one aqueous gelling agent, and to the uses of the article, before and after being moistened with water, for example in the cosmetic or dermatological field, in particular for caring for, cleansing and/or removing makeup from the human skin, more especially the face, body and the eyes.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out. As used in the above description of the invention the phrase “selected from the group consisting of” includes plural members of the group regardless whether the phrases “at least one” and/or “and mixtures thereof” is used in combination therewith.

What is claimed is:

1. An article comprising:
(A) a water-insoluble substrate comprising one or more layers, and, in contact therewith,
(B) a substantially anhydrous composition comprising at least 10% by weight of one or more oils relative to the total weight of the composition, at least one emulsifying surfactant, and at least one hydrophilic gelling agent.

2. The article according to claim 1, wherein the composition comprises less than 5% by weight of water, relative to the total weight of the composition.

3. The article according to claim 1, wherein the hydrophilic gelling agent comprises one or more hydrophilic polymers.

4. The article according to claim 1, wherein the hydrophilic polymer exists in the form of a powder or in the form of a water-in-oil emulsion.

5. The article according to claim 3, wherein the hydrophilic polymer is selected from the group consisting of crosslinked polymers of acrylic acid or methacrylic acid, polymers of 2-acrylamido-2-methylpropanesulphonic acid, crosslinked copolymers of acrylamide and 2-acrylamido-2-methylpropanesulphonic acid, and mixtures thereof.

6. The article according to claim 1, wherein the hydrophilic gelling agent is present in an amount of 0.1 to 20% by weight relative to the total weight of the composition.

7. The article according to claim 1, wherein the composition comprises 10 to 99% by weight oil relative to the total weight of the composition.

8. The article according to claim 1, wherein the composition comprises 0.1 to 90% by weight surfactant relative to the total weight of the composition.

9. The article according to claim 1, wherein the surfactant has an HLB of 5 to 15.

10. The article according to claim 1, wherein the surfactant is a nonionic surfactant selected from the group consisting of fatty acid esters of polyols and their oxyalkylenated derivatives; fatty alcohol ethers of polyols and their oxyalkylenated derivatives, and mixtures thereof.

11. The article according to claim 10, wherein the nonionic surfactant is selected from the group consisting of oxyethylenated sorbitan fatty acid esters, oxyethylenated glyceryl fatty acid esters, polyglyceryl fatty acid esters, polyethylene glycol fatty acid esters, polyoxyethylenated and/or polyoxypropylenated fatty alcohol ethers, and mixtures thereof.

12. The article according to claim 1, wherein the composition comprises (i) from 1 to 10% by weight of one or more hydrophilic gelling agents selected from the group consisting of crosslinked polymers of acrylic acid or methacrylic acid, polymers of 2-acrylamido-2-methylpropanesulphonic acid, crosslinked copolymers of acrylamide and 2-acrylamido-2-methylpropanesulphonic acid; (ii) from 30 to 90% by weight of one or more oils; and (iii) from 5 to 40% by weight of one or more emulsifying surfactants selected from the group consisting of fatty acid esters of polyols and their oxyalkylenated derivatives; fatty alcohol ethers of polyols and their oxyalkylenated derivatives.

13. The article according to claim 1, wherein the composition further comprises at least one adjuvant selected from the group consisting of organic solvents, emollients, antioxidants, chelators, perfumes, screening agents, colouring matter, hydrophilic or lipophilic active agents, lipophilic gelling agents, lipid vesicles, and mixtures thereof.

14. The article according to claim 13, wherein the adjuvant is a lipophilic gelling agent.

15. The article according to claim 1, wherein the composition has a viscosity of less than 150 mPa.s.

16. The article according to claim 1, wherein the substrate is selected from the group consisting of woven materials, nonwoven materials, foams, sponges, cotton wool, and mixtures thereof.

17. The article according to claim 1, wherein the substrate is a nonwoven material comprising fibers of natural origin or of synthetic origin.

18. The article according to claim 1, wherein it comprises 10 to 1500% by weight of composition relative to the weight of the substrate.

19. The article according to claim 1, in the form of a rectangular wipe, a glove, a mitten or a round compress.

20. The article according to claim 1, wherein said article is an article for caring for and/or treating the skin.

21. The article according to claim 1, wherein said article is an article for cleansing and/or removing makeup from the skin and/or the eyes.

22. A method for cleansing and/or removing makeup from the skin and/or the eyes comprising contacting said skin and/or eyes with an article according to claim 1 which has been contacted with water.

23. The method according to claim 22, wherein said skin and eyes are sensitive skin and sensitive eyes.

24. A process comprising adding to or impregnating into a water-insoluble substrate comprising one or more layers a substantially anhydrous composition comprising at least 10% by weight of one or more oils, relative to the total weight of the composition, at least one emulsifying surfactant, and at least one hydrophilic gelling agent.

25. An article made by the process according to claim 24.

26. A method for cleansing and/or removing makeup from the skin and/or the eyes comprising contacting said skin and/or eyes with an article according to claim 25 which has been contacted with water.

27. The method according to claim 26, wherein said skin and eyes are sensitive skin and sensitive eyes.

28. A method comprising:
moistening an article comprising:
(A) a water-insoluble substrate comprising one or more layers, and, in contact therewith,
(B) a substantially anhydrous composition comprising at least 10% by weight of one or more oils relative to the total weight of the composition, at least one emulsifying surfactant, and at least one hydrophilic gelling agent with water; and
causing water to penetrate and emulsify the composition.

29. The method as claimed in claim 28, wherein said compositin forms a creamy texture upon causing water to penetrate and emulsify the composition.

30. An article made by the process according to claim 28.

31. A method for cleansing and/or removing makeup from the skin and/or the eyes comprising contacting said skin and/or eyes with the article according to claim 30.

32. The method according to claim 31, wherein said skin and eyes are sensitive skin and sensitive eyes.
